(19) United States
(12) Patent Application Publication
(10) Pub. No.: US 2015/0050326 A1
(43) Pub. Date: Feb. 19, 2015

(54) METHOD FOR THE REALIZATION OF A BIOCOMPATIBLE BONE IMPLANT COMPRISING GRANULAR ELEMENTS AND A BIOREABSORBABLE BIOPOLYMER GEL AND BIOCOMPATIBLE BONE IMPLANT THUS OBTAINED

(71) Applicant: EUROCOATING S.p.A., PERGINE VALSUGANA (TRENTO) (IT)

(72) Inventors: Francesco Bucciotti, Civezzano (TN) (IT); Marzio Piccinini, Lavarone (TN) (IT); Susanna Prosperi, Trento (TN) (IT)

(21) Appl. No.: 14/384,977

(22) PCT Filed: Mar. 14, 2013
(86) PCT No.: PCT/IB2013/052028

§ 371 Date: Sep. 12, 2014

(30) Foreign Application Priority Data
Mar. 14, 2012 (IT) VR2012A000045

(51) Int. Cl. A61L 27/34; A61L 27/58; A61L 27/54; A61L 27/02; A61L 27/50

(52) U.S. Cl. CPC A61L 27/34; A61L 27/02; A61L 27/50; A61L 27/54; A61L 27/58; A61L 2420/02; A61L 2430/02
USPC 424/426; 623/23.51; 427/2.14; 424/93.1; 424/529

(57) ABSTRACT

Method for providing a biocompatible bone implant adapted to fill a bone cavity or lacuna including the steps of providing granular elements in form of granules and/or granule aggregates, providing at least one primary polymeric solution including a biopolymer, at least partly coating the granular elements with the primary polymeric solution, obtaining coated granular elements including a biopolymer coating, providing a cross-linking solution, dipping the coated granular elements in the cross-linking solution, waiting a period of time referred to as “jellification time” of between 30 seconds and 1 hour, in which during said waiting step there occurs the cross-linking of the biopolymer coating of the granular elements with the cross-linking solution and the jellification of the coating, with the aim of obtaining a cohesive biocompatible implant including granular elements and a bio-reabsorbable biopolymer gel; biocompatible bone implant obtained through such method.

### TECHNICAL FIELD OF THE INVENTION

The present invention regards a method for the realization of a biocompatible bone implant comprising granular elements and a reabsorbable biopolymer material made of gel.

Furthermore, the present invention regards a biocompatible bone implant obtained according to such method.

### STATE OF THE ART

In the field of biomaterials, in particular of reabsorbable biomaterials that can be used in the biomedical area, especially in the orthopaedic and surgical sectors, ceramic and/or polymer biomaterials are currently mostly utilized, substantially used for fixing prosthesis or as bone replacements, for filling cavities or lacunae of various origin and type.

The use of calcium-phosphate-based ceramics in the bone regeneration, in form of injectable micro-particles, has considerably developed over the last years due to the good biological response obtained by such materials and the suitable mechanical properties thereof.

Furthermore, the calcium phosphate-based compounds are known and used in biomedical applications due to the chemical composition thereof similar to the mineral inorganic fraction of the bone tissue, a characteristic that makes them biocompatible, bioactive and osteoconductive, in the sense that they stimulate bone regeneration and the molecular ionic exchange with the tissue.

Patent application U.S. 2005/0209704 describes a biocompatible and biodegradable implant that can be adapted to the bone cavities made up of granules comprising biopolymers, bioceramics, bioglasses, etc., and comprising at least one layer of coating made of a biodegradable and biocompatible polymer by dipping or spray coating.

Such coated granules are “fused” together through a polymer connection of the polymeric coating of granules close to each other to obtain the implant; the methods through which the fusion occurs are substantially the thermal treatment and the use of pressurized CO₂ which serves as a solvent for the polymeric coating.

There are also present other types of bone implants comprising granules. In one of these there are granules coated with polylactic acid and an activator which determines the formation of an adhesive matrix which joins the various granular elements. However, upon performing the implantation, such adhesive matrix solidifies upon contact with blood.

Patent application WO 2009/073068 describes a method for obtaining a bone implant made up of calcium phosphate particles coated/functionalised with alkoxysilanes. The alkoxysilane coating forms a connection structure with the particles themselves and a polyamide polymeric material matrix. Such connection structure is covalently bonded both to calcium phosphate and to the matrix.

Patent application WO 2005/084725 describes a bone implant made up of bioactive particles coated with a coating. The particles may be made of calcium phosphate or demineralised bone, the coating consists of a polymeric material, for example gelatine or alginate. The composition to be implanted into the bone cavity is wetted with an aqueous or saline solution.

U.S. Pat. No. 5,085,861 describes an implant that can be adapted to the bone cavities, or a bone cement, made up of particles made of calcium phosphate incorporated in a biodegradable and cross-linked polyester matrix. Patent application WO 2012/007917 describes an implant made up of a composite material comprising pectin and a calcium phosphate powder.

### SUMMARY OF THE INVENTION

Thus, the technical task of the present invention is to improve the prior art.

Providing a method for the realization of a biocompatible bone implant adapted to fill cavities or bone lacunae and serving as bone substitute interfacing with the organic tissues without creating adverse reactions in the tissue or at systemic level represents an object of the present invention.

Another object of the present invention is to provide a method for the realization of a biocompatible bone implant with a good mechanical stability in the first grafting period given by the bio-reabsorbable biopolymer gel, required for the bone regeneration given that it avoids the micro-displacements of the granular elements.

A further object of the present invention is to provide a method for the realization of a bone implant biocompatible with the tissue with which it is interfaced and useable in the bone regeneration field, as a bone filler and for releasing drugs or other substances, etcetera.

A further object of the present invention is to provide a method that is simple to obtain, with the possibility of using the materials currently available on the market.

Still, a further object of the present invention is to provide a method that is inexpensive.

These and other objects are attained by a method for the realization of a biocompatible bone implant according to the present principles.

Within such technical task, providing a biocompatible bone implant adapted to be interfaced with the organic tissues without creating adverse reactions in the tissue or at system level represents an object of the present invention.

Another object of the present invention is to provide a biocompatible bone implant with a good mechanical stability in the first grafting period given by the bio-reabsorbable biopolymer gel, required for the bone regeneration due to the fact that it avoids micro-displacements of the granular elements.

A further object of the present invention is to provide a biocompatible bone implant with the tissue with which it is interfaced and useable in the bone re-generation field, as a bone filler and for releasing drugs or other substances etc.

Still another object of the present invention is to provide a biocompatible bone implant that is simple to obtain, with the possibility of using materials currently available on the market.

These and other objects are attained by a biocompatible bone implant according to the present principles.

### DETAILED DESCRIPTION OF THE INVENTION

In the present document, the term biomaterial is used to indicate a biocompatible material capable of being interfaced with a biological system with the aim of increasing, treating or replacing any tissue, organ or function of the system.

The present invention refers to a method for providing biocompatible bone implant to be used as a bone replacement for filling a bone cavity or lacuna. The bone implant comprises granular elements in form of granules and/or granule aggregates and a gel polymeric material.

Furthermore, the present invention refers to the biocompatible bone implant obtained through the aforementioned method.

The granular elements present in the biocompatible bone implant comprise bioceramic, or biopolymer, or bioglass, or metal materials and/or a mixture thereof.

The bioceramics comprise calcium sulphates, calcium carbonates, calcium phosphates with Ca/P molar ratio comprised between 1.0 and 2.0, such as for example hydroxyapatite (HA), and/or beta-tricalcium phosphate (BTCP) and/or tetracalcium phosphate (TTCP) and/or alpha tricalciumphosphate and/or mixtures thereof. Such bioceramics are re-absorbable.

The metals may comprise titanium, titanium alloys, other metals suitable for the purpose and/or mixtures thereof.

The granular elements are dense and porous with open, closed and/or interconnected pores.

The granular elements of the present invention have a regular shape which provides a large area of contact between one granule and the other, such as for example the spherical shape.

However, the granular elements may have other shapes without departing from the scope of protection of the present invention.

The granular elements may have irregular shapes.

The method for the realization of a biocompatible bone implant according to the present invention comprises the step of providing granular elements in form of granules and/or granule aggregates.

The granular elements of the biocompatible bone implant have a grain size of diameter comprised between 10 and 5000 microns. The grain size and the type of granular elements may vary according to the interconnected intergranular macroporosity intended to be obtained in the implant.

Actually, the larger the dimension of the granular elements, the larger the interconnected macroporosity. In a variant of the invention, the granular elements of the biocompatible bone implant have the same grain size.

In a further variant, the grain size of the granular elements which form the biocompatible bone implant differs from one granule to the other. This allows varying the macroporosity of the implant by selecting granular elements with different grain size.

In a variant of the method of the present invention, two or more different grain sizes may be used for obtaining an interconnected porosity that is more diverse and with a larger distribution of holes.

In a non-limiting example of the invention, a biocompatible bone implant may comprise granules measuring 5000 microns of diameter, which provide a given macroporosity. Such macroporosity, present between the granules of 5000 microns of diameter, may be substantially filled by granules of dimensions equivalent to 300 microns, contained in the same implant. Thus, the overall interconnected macroporosity reduces given that the average diameter of the interconnected pores forming the macroporosity is reduced.

The average diameter of the interconnected macroporosity is comprised between 5 and 500 microns; the interconnected macroporosity comprises and it is also given by the presence of the jellified biopolymer between the granules.

The macroporosity between the granules is formed after the dissolution of the biopolymer.

Furthermore, even the porosity of the granules is made available following the dissolution of the biopolymer.

Such dissolution of the biopolymer consists in the breaking of the cross-linking points and it is followed by the degradation which consists in the breaking of the free chains of the biopolymer.

The dissolution of the biopolymer occurs quickly. In particular, the dissolution of the biopolymer is much more rapid than the bone re-absorption of the granular element, when the latter are made of re-absorbable material and when not dealing with non-re-absorbable elements such as titanium.

The method according to the present invention allows identifying and selecting a type of granular elements, in terms of chemical/physical composition, grain size, shape, and selecting granules and/or granule aggregates, to obtain different speeds and possibilities of re-absorption of the implant.

The polymeric material comprises a natural or synthetic biocompatible and bio-reabsorbible biopolymer. The biopolymer is selected from among polysaccharides, alginate, chitosan, pectin, chitin, etc.

In particular, alginate and chitosan have anti-inflammatory properties.

The method according to the present invention comprises the step of providing at least one primary polymeric solution comprising an aqueous component and a biopolymer, wherein the polymeric material is dissolved to form a polymeric aqueous solution.

The at least one primary polymeric solution has a biopolymer concentration comprised between 0.01% and 20% in weight.

The method according to the present invention comprises the step for coating the granular elements with a coating obtained using at least one primary polymeric solution and obtaining coated granular elements comprising a biopolymer coating.

The coating step occurs through the steps of soaking the granular elements with the at least one primary polymeric solution by dipping or spraying or spreading or any other method suitable for the purpose and drying the soaked coated granular elements, obtaining coated granular elements with a biopolymer coating. In the drying step there occurs the elimination of the aqueous component of the at least one primary polymeric solution and the deposition of the biopolymer of the at least one primary polymeric solution on the granular element, obtaining a dried granular element coated with biopolymer.

The thickness of the coating of biopolymer is comprised between 0.1 microns and 200 microns or between 1 micron and 200 microns or between 5 microns and 100 microns or between 0.1 microns and 100 microns or between 5 microns and 200 micron. Thus, there is obtained a thick coating, similar to a film which covers the granular elements fully or partly.

Following the addition of the cross-linking solution, as better described hereinafter, there is obtained a gel capable of filling all the spaces between one granule and the other and which allows, during the implant, not leaving the bone implant empty and thus preventing the implant from collapsing.

The step of drying the granular elements occurs over a period of time comprised between 5 minutes and 12 hours; the drying step occurs at a drying temperature lower than 100° C., so as to eliminate the entire aqueous component and not degrade the biopolymer. In a variant of the invention, the drying temperature is 37° C. The percentage of aqueous component that is eliminated is comprised between 85% and 100%; the drying step occurs in an oven or in any other device suitable for the purpose.

During the drying step, the coating of biopolymer which covers the granular element determines the aggregation of the granular elements. Thus the drying step is followed by the steps of separating and sieving the aggregated coated granular elements. The separation step occurs through manual or automatic breaking; the sieving step occurs through manual or mechanical screening with the aim of obtaining a given grain size of the dried coated granular elements and substantially selecting the initially intended grain size of the granular elements.

After the sieving step, the biopolymer coating has a thickness comprised between 0.1 and 200 microns to maintain good cohesiveness between the granular elements sufficient for the in situ placement of the biocompatible bone implant and stabilising the granular elements; the weight inorganic/organic ratio of the coated granular elements is lower than 1. Actually, during the first period after grafting of the bone implant according to the present invention, the re-absorbable biopolymer gel confers good mechanical stability to the implant, required for bone regeneration, given that it prevents the micro-displacements of the granular elements.

The method according the present invention further comprises a step for providing a cross-linking solution and a step for dipping the coated granular elements in the cross-linking solution.

The cross-linking solution is selected from among a solution comprising a salt, such as for example calcium chloride or any other salt, or a solution containing bivalent ions such as Ca, Zn, Sr or a solution containing a bio-reabsorbable natural polymer such as for example chitosan or chitin or any other polymer suitable for the purpose, or a saline solution or a buffer solution or blood solution or containing cells or mixtures thereof.

The cross-linking solution, in a variant of the invention, comprises drugs, cell growth factors, cells or mixtures thereof. These substances are made available during the dissolution of the gel which, as outlined further in detail hereinafter, thus serves as a carrier for the same.

Zinc, for example, is useful for its antibacterial properties; strontium is selected for its anti-osteoporotic usefulness.

The aforementioned saline solutions have a molar concentration comprised between 0.001 and 3 M or between 0.01 and 3 M or preferably comprised between 0.01 and 0.5 M or between 0.001 and 0.5 M; the aforementioned polymeric solutions have a polymer concentration comprised between 0.001% and 5%.

In addition, there is present a step of identifying a suitable ratio between the volume of the coated granular elements (inorganic material) and the volume of the cross-linking solution. Such ratio is greater than 1 to ensure that the coated granular elements are completely wetted by the cross-linking solution.

The method further comprises a waiting step for a period of time referred to as “jellification time” comprised between 30 seconds and 1 hour, during which the coated granular and dried elements remain dipped in the cross-linking solution.

During the jellification time there occurs the hydration and/or the cross-linking of the coating biopolymer of the granular elements with the cross-linking solution and the jellification of the coating, so as to obtain a cohesive biocompatible bone implant comprising granular elements and a bio-reabsorbable biopolymer gel. Actually, the biopolymer of the coating of the granular elements is hydrated by the cross-linking solution with ensuing cross-linking of the same. This allows forming a continuous gel which keeps the granular elements cohesive and constrained therebetween. The hydration of the biopolymer occurs instantaneously, upon contact of the cross-linking solution with the coated granular elements and dried.

In a variant of the invention, the process occurs at room temperature.

After a few minutes, the method comprises a step of eliminating the excess cross-linking solution, i.e. the one that has not reacted with the biopolymer cross-linking it.

In a variant of the invention, the method comprises a step of introducing the dried coated granular elements in an injection device. Such injection device, in a non-limiting embodiment of the present invention, comprises a syringe with a larger opening than the average diameter of the single granular elements.

The method comprises a step of packing the granular elements introduced into the injection device through manual pressure or any other packing suitable for the purpose.

There is present a step of dipping the granular elements in the cross-linking solution which occurs through the step of introducing the cross-linking solution into the injection device. After a few minutes, once the jellification occurs, there occurs the step of extruding the compound comprising granular elements and a re-absorbable biopolymer gel. This extrusion occurs in a bone cavity, obtaining a biocompatible bone implant. The re-absorbable bone implant comprising granular elements and the extruded re-absorbable biopolymer gel is adapted to and maintains the shape of the bone cavity in which it is extruded.

The biopolymer gel, in the composition in which it comprises a natural polysaccharide, has inherent anti-inflammatory properties and it may accelerate the healing of the implant site after the intervention. Furthermore, such bone implant may be doped using zinc, which serves as an anti-bacterial agent, and using strontium, which serves as an osteoporotic agent, thus acquiring the properties of such agent.

Furthermore, the gel limits the loss/migration of the granules outside the bone cavity during the steps of implantation.

Furthermore, the biopolymer gel which confers a good initial stability to the implant avoiding the use of membranes which complicate the intervention, may be cause of further inflammatory processes and eventually make the surgery site “dirty”. Actually, the gel, being stable at contact with ionic solutions and/or biological fluids, may be implanted within the cavity or bone lacuna without being subjected to immediate solubilisation and/or degradation phenomena. Therefore, the bio-reabsorbable biopolymer gel has a composition such not to be degraded and/or solubilised by the contact with biological fluids and/or ionic solutions immediately after the implantation thereof, and it is thus capable of maintaining the bone implant in situ over a determined period of time.

For example sodium alginate, after cross-linking with the cross-linking solution, is not soluble in ionic solutions.

The bio-reabsorbable polymer gel is initially inert to the biological environment with which it comes into contact.

The bio-reabsorbable polymer gel is capable of remaining in gel form, adapted to maintain the cohesion of the implant, even after the introduction thereof in the site subject of implantation and after coming to contact with biological fluids and/or ionic solutions. The role of the bio-reabsorbable biopolymer gel is mainly related to maintaining the cohesion, at least initial, of the bone implant. Furthermore, the biopolymer gel allows improving the malleability of the product.

The processes of reabsorbing the cross-linked gel are mainly guided by metabolic processes which provide for the degradation of gel over time. In case of inclusion of drugs, cell growth factors, cells or mixtures thereof, carried within the gel through the cross-linking solution, the same shall not be made immediately available but they shall be released over time with a kinetic release depending on the reabsorption of the gel.

The bone implant comprising granular elements and the extruded bio-reabsorbable biopolymer gel may be modelled to directly fill the bone cavity, simultaneously maintaining the cohesion of the granular elements in water and in the saline solution at the temperature of 37° C.

The total volume occupied by the implant according to the present invention is made up of the volume of the granular elements and the volume of the biopolymer gel between the granular elements. The percentage of the volume occupied by the granular elements is comprised between 1% and 99%. The granular elements are physically maintained together and cohesive by the biopolymer gel. Upon completing the implantation, the gel does not form a solid body at contact with blood or biological fluids but remains in jellified form and reabsorbed progressively.

Further advantages conferred by the present invention lie in the fact that the jellification step occurs mainly outside the surgery site and thus prevent possible allergic reactions to reaction products, different from what occurs when the triggering of the jellification is caused by biological or blood fluids, this being a case in which such allergy or adverse situations may occur.

Furthermore, in such situation, the triggering may not occur for genetic reasons or due to lack of specific chemical elements.

The jellification step according to the present invention confers safety to the implant, improving the stability thereof and guaranteeing the occurrence of jellification, not directly involving the fluids of the patient.

The consistency of the polymer gel according to the present invention is substantially viscous in an initial stage; it tends to harden progressively over time, in particular if wet by bivalent ions, like calcium, present in the saline solutions. However, the initial viscous consistency confers the gel sufficient rigidity, before grafting thereof, thus not jeopardising the stability of the implant.

The biopolymer coating is present in the porosity present in the granular elements.

The method according to the present invention further comprises a step for sterilising the components used in the various method steps or the granular elements or obtained materials. The sterilization step occurs through gamma rays, beta rays, ethylene oxide, autoclaves or other methods suitable for the purpose.

The granular elements are pure or doped with ions such as zinc, strontium and magnesium and/or a mixture thereof; the step of providing the granular elements occurs by using granules and/or granule aggregates pure or doped with ions such as zinc, strontium and magnesium and/or a mixture thereof. The doping substances confer improved biological properties to the granular elements. The role of the doping agents is very important for improving the osteo-integration of the bone substitute and the regeneration of the very bone. For example, zinc has anti-bacterial properties; strontium instead has anti-osteoporotic properties. It is important to verify the chemical composition of the doped granular element to avoid excess concentration of doping agents, which could be toxic for the organism (for example, a percentage of ZnO greater than 1.5% in weight).

It has thus been observed that the invention attains the proposed objects.

The present invention has been described according to preferred embodiments but equivalent variants may be conceived without departing from the scope of protection outlined by the claims that follow.

Furthermore, some of the characteristics described for a variant of the present invention may be combined with characteristics defined for a different variant, without departing from the scope of protection of the present invention.

1. A method for the realization of a biocompatible bone implant adapted to fill a bone cavity or lacuna comprising the following steps:
providing granular elements in form of granules and/or granule aggregates,
providing at least one primary polymer solution comprising a biopolymer,
coating said granular elements with said primary polymer solution,
obtaining coated granular elements comprising a biopolymer coating,
providing a cross-linking solution,
immerging said coated granular elements in said cross-linking solution, waiting for a time period defined as “gelification time” ranging from 30 seconds to one hour,
wherein during said waiting step the cross-linking of said biopolymer coating of said granular elements by means of said cross-linking solution and the gelification of said coating occur, in order to obtain a cohesive biocompatible implant comprising said granular elements and a bioreabsorbable biopolymer gel.

2. The method according to claim 1, wherein said step of providing granular elements occurs by selecting granules and/or granule aggregates comprising at least one among a bioceramic material, comprising calcium sulphate, calcium carbonate, calcium phosphate with Ca/P molar ratio ranging from 1.0 to 2.0, hydroxyapatite (HA), beta-tricalcium phosphate (BTCP) and/or tetracalcium phosphate (TTCP) and/or alpha tricalcium phosphate and/or mixtures thereof, a biopolymer, a bioglass, a metal, titanium, titanium alloys and/or a mixture thereof.

3. The method according to claim 1, wherein said step of providing granular elements is carried out by selecting granules and/or granule aggregates doped with zinc, strontium, magnesium ions and/or a mixture thereof.

4. The method according to claim 1, wherein said step of providing granular elements is carried out by selecting granules and/or granule aggregates with a grain size ranging from 10 to 5000 microns.

5. The method according to claim 1, further comprising a step of individuating a ratio greater than 1 between volume of said coated granular elements and volume of said cross-linking solution for ensuring that said coated granular elements are completely imbibed by said cross-linking solution.

6. The method according to claim 1, further comprising a step of sterilizing said granular elements and/or said primary polymer solution and/or said cross-linking solution and/or said coated granular elements by gamma rays, beta rays, ethylene oxide, autoclave or other methods suitable for the purpose.

7. The method according to claim 1, wherein said step of providing at least a primary polymer solution is carried out by selecting said biopolymer among natural or synthetic biocompatible and bioreabsorbable biopolymers, polysaccharides, alginate, chitosan, pectin, chitin, at a biopolymer concentration ranging from 0.01% to 20% in weight.

8. The method according to claim 1, wherein said step of coating said granular elements with said primary polymer solution comprises the following steps:
imbibing said granular elements with said primary polymer solution by immersing or spraying or spreading or any other method suitable for the purpose,
drying said imbibed granular elements, thus obtaining coated granular elements comprising a biopolymer coating.

9. The method according to claim 8, wherein said step of drying said coated granular elements is carried out at a drying temperature lower than 100° C. for a time ranging from 5 minutes to 12 hours.

10. The method according to claim 1, wherein said step of coating said granular elements with said primary polymer solution comprises at least partially imbibing said granular elements with said primary polymer solution by immersing or spraying or spreading or any other method suitable for the purpose.

11. The method according to claim 1, comprising a step of separating and/or sieving said coated granular elements, wherein said step of separating said coated granular elements occurs by manual or mechanical crushing and/or wherein said step of sieving said coated granular elements occurs by manual or mechanical screening in order to obtained a given grain size.

12. The method according to claim 1, wherein said biopolymer coating has a thickness ranging from 0,1 micron to 200 microns or ranging from 1 micron to 200 microns or ranging from 5 microns to 100 microns or ranging from 0,1 micron to 100 microns or ranging from 5 microns to 200 microns and said coated granular elements have a weight inorganic/organic ratio lower than 1.

13. The method according to claim 1, wherein said step of providing a cross-linking solution occurs by selecting a solution comprising calcium chloride or another salt suitable for the purpose, a solution containing divalent ions such as Ca, Zn, Sr or a solution containing chitosan or chitin or a bioreabsorbable natural polymer or saline solutions, or buffer solutions, or blood solutions or solutions containing cells, drugs, cellular growth factors, or mixtures thereof at a molar concentration of salts ranging from 0.01 to 3 M or ranging from 0.001 to 3 M and/or to a polymer concentration ranging from 0.001% to 5% in weight.

14. The method according to claim 1, wherein said step of providing a cross-linking solution occurs by selecting a solution comprising calcium chloride or another salt suitable for the purpose, a solution containing divalent ions such as Ca, Zn, Sr or a solution containing chitosan or chitin or a bioreabsorbable natural polymer or saline solutions, or buffer solutions, or blood solutions or solutions containing cells, drugs, cellular growth factors or mixtures thereof at a molar concentration of salts ranging from 0.01 to 0.5 M or ranging from 0.001 to 0.5 M.

15. The method according to claim 1, comprising a step of eliminating the excess cross-linking solution.

16. A biocompatible bone implant adapted to fill a bone cavity or lacuna, comprising granular elements in form of granules and/or granule aggregates and a bioreabsorbable biopolymer gel, wherein said granular elements are cohesive in said bioreabsorbable biopolymer gel.

17. The biocompatible bone implant according to claim 16, wherein said bioreabsorbable biopolymer gel has a composition such that it is not degraded and/or solubilised by the contact with biological fluids and/or ionic solutions immediately after its implant, and therefore it is able to maintain the bone implant in situ for a given time period.

18. The biocompatible bone implant according to claim 16, wherein said granular elements comprise at least one between a bioceramic material comprising calcium sulphate, calcium carbonate, calcium phosphate with Ca/P molar ratio ranging from 1.0 to 2.0, hydroxyapatite (HA), beta-tricalcium phosphate (BTCP) and/or tetracalcium phosphate (TTCP) and/or alpha-tricalcium phosphate and/or mixtures thereof, a biopolymer, a bioglass, a metal, titanium, titanium alloys and/or a mixture thereof.

19. The biocompatible bone implant according to claim 16, wherein said granular elements are doped with zinc, strontium, magnesium ions and/or a mixture thereof.

20. The biocompatible bone implant according to claim 16, wherein said granular elements have a grain size ranging from 10 to 5000 microns.

21. The biocompatible bone implant according to claim 16, wherein said bioreabsorbable biopolymer gel is obtained from the gelification of a biopolymer coating of said granular elements, comprising a biopolymer selected from among natural or synthetic biocompatible and bioreabsorbable biopolymers, polysaccharides, alginate, chitosan, pectin, chitin, at a biopolymer concentration ranging from 0.01% to 20% in weight, wherein said cross-linking solution comprises a solution comprising calcium chloride or another salt, or divalent ions such as Ca, Zn, Sr or chitosan or chitin or a natural bioreabsorbable polymer, or a saline solution, or a buffer solution, or a blood solution or a solution containing cells, drugs, cellular growth factors or mixtures thereof at a molar concentration of salts ranging from 0.01 to 3 M or ranging from 0.001 to 3 M or at a concentration of polymers ranging from 0.001% to 5% in weight.

22. The biocompatible bone implant according to claim 16, wherein said biopolymer coating of said granular elements, comprising a biopolymer selected from among natural or synthetic biocompatible and bioreabsorbable biopolymers, polysaccharides, alginate, chitosan, pectin, chitin, at a biopolymer concentration ranging from 0.01% to 20% in weight, wherein said cross-linking solution comprises a solution comprising calcium chloride or another salt, or divalent ions such as Ca, Zn, Sr or chitosan or chitin or a natural bioreabsorbable polymer, or a saline solution, or a buffer solution, or a blood solution or a solution containing cells, drugs, cellular growth factors or mixtures thereof at a molar concentration of salts ranging from 0.01 to 0.5 M or ranging from 0.001 to 0.5 M.

23. The biocompatible bone implant according to claim 16, wherein said biopolymer coating has a thickness ranging from 0,1 to 200 microns or ranging from 1 micron to 200 microns or ranging from 5 microns to 100 microns or ranging from 0,1 micron to 100 microns or ranging from 5 microns to 200 microns.
